(19) United States
(12) Patent Application Publication
(10) Pub. No.: US 2007/0036838 A1
(43) Pub. Date: Feb. 15, 2007

(54) Methods and compositions for soothing oral and nasal tissues

(75) Inventors: Siobhan Mary Keenan, Ascot (GB); Jenny Elizabeth Bermin, Staines (GB); Kristofer Tonstrom, Stockholm (SE)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/499,997

(22) Filed: Aug. 7, 2006

(30) Foreign Application Priority Data
Aug. 12, 2005 (EP) 05017601.5

(51) Int. Cl. A61K 31/445; A61K 31/401; A61K 9/68

(52) U.S. Cl. 424/440; 514/315; 514/423

(57) ABSTRACT

Use of a salivating agent in the preparation of a medicament for soothing irritated oral and/or nasal tissues is provided. Compositions comprising a salivating agent and a cooling agent are also provided. The use and compositions provided herein provide improved soothing and relief of irritated oral and/or nasal tissues.

### FIELD OF THE INVENTION

The present invention relates to the use of salivating agents for the preparation of a medicament for soothing irritated oral and/or nasal tissues.

### BACKGROUND OF THE INVENTION

Many examples exist of medicaments designed to relieve or sooth irritated oral and nasal tissues. These medicaments typically rely upon a pharmaceutically active ingredient such as benzocaine which is a local anaesthetic. These ingredients, whilst on the whole successful, are sometimes not able to effectively sooth the nasal tissues and the nasopharyngeal region. Furthermore, the effects of pharmaceutically active ingredients such as these are sometimes considered by consumers as being more than is necessary to achieve the required results. A need exists for medicaments that provide a soothing effect in a moderate manner.

Soothing of irritated oral and nasal tissues may occur via increased moisturisation of the tissues. However, many ingredients suitable for increasing moisturisation of the oral and nasal tissues have noted drawbacks. For example, citric acid is able to increase oral salivation around the area of the tongue; yet is not able to improve moisturisation in the entirety of the oral cavity and does not modulate nasal moisturisation. This tends to prevent citric acid from being able to provide notable soothing effect in the nasophayngeal area. Furthermore, levels of citric acid that are capable of producing noticeable benefits around the tongue may taste acidic or astringent, and may also negatively impact product stability due to the high hygroscopicity of citric acid.

A need exists to provide medicaments that successfully sooth irritated oral and/or nasal tissues without associated aesthetic negatives and/or product stability issues.

### SUMMARY OF THE INVENTION

The present invention provides medicament compositions that effectively sooth irritated oral and/or nasal tissues. The medicaments herein incorporate a salivating agent that modulates oral and/or nasal secretions, providing relief to a consumer's oral and/or nasal tissues. The invention provides for the use of a salivating agent according to formula (I):

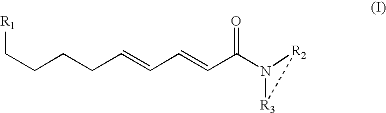

wherein R₁ represents C1-C2 n-alkyl; R₂ is 2-methyl-1-propyl and R₃ is hydrogen, or R₂ and R₃ taken together is a moiety having the formula —(CH₂)ₙ— wherein n is 4 or 5, or mixtures thereof in the preparation of a medicament for soothing irritated oral and/or nasal tissues by modulating oral and/or nasal secretions. The invention further provides a composition for modulating oral and/or nasal secretion comprising:
a) a salivating agent according to formula (I): 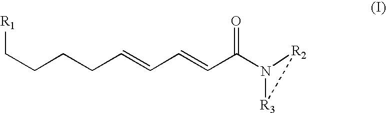
wherein R₁ represents C1-C2 n-alkyl; R₂ is 2-methyl-1-propyl and R₃ is hydrogen, or R₂ and R₃ taken together is a moiety having the formula —(CH₂)ₙ— wherein n is 4 or 5, or mixtures thereof, and
b) a cooling agent comprising menthol, peppermint oil, N-substituted-p-menthane -3-carboxamides, acyclic tertiary and secondary carboxamides, 3-1-menthoxy propan-1,2-diol, monomenthyl glutarate and mixtures thereof.

### DETAILED DESCRIPTION OF THE INVENTION

Unless otherwise stated herein, all percentages are weight percentages.

Unless otherwise stated herein, all measurements are taken at 25° C.

The present invention provides the use of a salivating agent in the preparation of a medicament for soothing irritated oral and/or nasal tissues by modulating oral and/or nasal secretion. As used herein, “oral” includes the buccal cavity, tongue and the throat, and “nasal” includes the nose, nasal cavity and the nasopharynx. As used herein, “soothing” includes relaxing, moisturizing, relieving, reducing pain and the like. Preferably, the medicament sooths irritated oral and nasal tissues by modulating oral and nasal secretions. Oral and nasal secretion includes salivation, moisturisation and mucosal secretion, preferably in the nasal passage, nasal conchae, pharynx, nasopharynx, oral cavity, soft palate and the tongue.

The salivating agent of the present invention comprises a material according to formula (I): 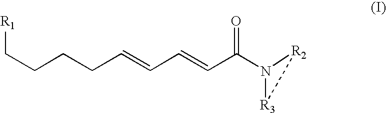
wherein R₁ represents C1-C2 n-alkyl; R₂ is 2-methyl-1-propyl and R₃ is hydrogen, or R₂ and R₃ taken together is a moiety having the formula —(CH₂)ₙ— wherein n is 4 or 5, or mixtures thereof. Preferably, the salivating agent comprises a material wherein R₂ is 2-methyl-1-propyl and R₃ is hydrogen, more preferably wherein R₁ is C1 n-alkyl, R₂ is 2-methyl-1-propyl and R₃ is hydrogen. More preferably, the salivating agent comprises trans-pellitorin, a chemical having a structure according to formula (II):

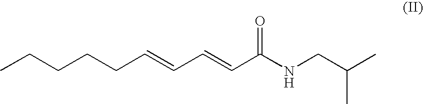

Methods of making the salivating agents suitable for use herein are described in US2004/0241312 A1 and EP1520850A2.

Without wishing to be bound by theory it is believed that the salivating agents herein have surprisingly been found to sooth and relieve irritated oral and/or nasal tissues when applied thereupon. It is believed that this effect is associated with the action of the salivating agents modulating oral and/or nasal secretions, whilst not impacting the aesthetics or stability of the medicament.

Furthermore, the salivating agents as described herein provide improved long-lasting moisturisation in the oral and/or nasal tissues, in comparison with organic acids such as citric acid. Without wishing to be bound by theory, this is believed to be due to two factors. In the first instance, it is believed that the lipophilic nature of the salivating agents herein enables the compounds to adhere to the mucosal tissues present in the oral and nasal tissues. This adherence enables continued stimulation of oral and/or nasal secretions, especially where the secretions themselves may wash the salivating agent away, for example such as is the case with organic hydrophilic acids such as citric acid. Furthermore, it is believed that the salivating agents herein actually stimulate oral and/or nasal secretions via nerve stimulation, rather than via osmotic pressure, as is the case with traditional salivating agents. Without wishing to be bound by theory, it is believed that the salivating agents herein stimulation secretion in the oral and/or nasal tissues via secondary processes transduced by interaction of trigeminal and parasympathetic nerves. Thus the salivating agents herein are only perceived in the oral cavity where they are ingested, but the combination of their lipophilic nature which enables increased residency time, and the stimulatory pathway, enables prolonged stimulation of secretion from both the oral and nasal tissues, driving improved moisturisation and soothing of the oral and nasal tissues. This is in comparison with traditional salivating agents that only stimulate oral secretion, and not driving relief and soothing in the nasal tissues.

Preferably, the medicament comprises from about 0.01% to about 5% salivating agent by weight of the medicament, preferably from about 0.01% to about 2%, more preferably from about 0.01% to about 1.5%, even more preferably from about 0.02% to about 1.0%.

The medicament of the present invention may be in any suitable form, including confectionery, chewing gum, throat and cough lozenge, throat disc, cough syrup and the like. Preferably the medicament is in the form of a confectionery. Suitable confectionery forms include hard boiled sweets, soft boiled sweets, chewing gums, gummy-based sweets, centre-fill confectionery, or lollies. The confectionery compositions of the present invention preferably take the form of a hard boiled sweet.

The compositions of the present invention are preferably in the form of a hard boiled candy or gum based confectionary. Hard boiled candies are sugar- or sugar substitute-based compositions wherein the base is formed into a candy mass with cooking and subsequently formed into a drop and allowed to cool. The candy mass once cooled forms a glassy matrix that contains the salivating agent therein. Once formed, the hard boiled candies preferably have a water level of about 0.1% to about 4% by weight of the composition. Gum based confectionary are soft to semi-solid compositions which are sugar or sugar-substitute based, wherein a suitable gelling agent is cooked with the sugar or sugar-substitute and water to achieve the right consistency, and either deposited into moulds or extruded into a continuous rope & cut.

A suitable sugar base for a hard candy comprises from about 30% to about 85% glucose syrup and from about 15% to about 70% sucrose. Alternatively, a sugar-free base can be used for the shell. Suitable sugar-free bases include bulk sweeteners such as isomalt, maltitol, sorbitol and xylitol. Isomalt and maltitol are preferred as bulk sugar-free bases. Xylitol is preferred as an ancillary base, preferably being present in sugar-free candies at a level of from about 0.1% to about 5%.

Other preferred forms of the medicament of the present invention are cough lozenges and cough syrups. Cough lozenges are sugar-based solid or semi-solid compositions, preferably in the form of hard boiled candies and/or gummies. A suitable cough lozenge comprises from about 30% to about 50% of glucose syrup and from about 15% to about 75% of sucrose. Cough lozenges may further comprise honey, honey derivatives and/or honey flavours or lenitive herbs. in concentration from about 0.05% to 10%, preferably from 0.1% to 5%.

Cough syrups are sugar-based liquid composition further comprising additional active ingredients, such guaifenesin. Cough syrups comprise from about 25% to about 65% of sucrose. Additionally, from about 30% to about 45% of glucose syrup may be further comprised in the formulation of cough syrups.

The medicament of the present invention may further comprise a cooling composition and/or a warming composition. The cooling and/or warming composition may be present in the medicament separately, or at the same time. When used at the same time, the cooling and warming compositions are preferably located in distinct and discrete regions within the medicament and are preferably adapted to provide sequential release profiles. As used herein, ‘adapted to provide sequential release profiles’ means that the compositions are chemically and/or physically modified relative to a homogeneous mix of the compositions. It will be understood that many such medicaments will release the warming or cooling agent over the period of ingestion of the product and that there may be some simultaneous perception of warming agent and cooling agent.

An essential component of the cooling composition is a physiological cooling agent. Suitable levels of the cooling agent are from about 0.001 to about 10%, preferably from about 0.01 to about 5%, more preferably from about 0.01 to about 2%, more preferably still from about 0.01 to about 0.5% by weight of the medicament. A test for physiological cooling agents is described in GB-A-1,452,291, published Oct. 13, 1976.

Suitable physiological cooling agents are described in WO97/06695. Preferred for use herein are physiological cooling agents selected from the group consisting of menthol, peppermint oil, N-substituted-p-menthane-3-carboxamides, acyclic tertiary and secondary carboxamides, 3-1-menthoxy propan-1,2-diol, monomenthyl glutarate and mixtures thereof. The carboxamides found most useful are those described in U.S. Pat. No. 4,136,163, Jan. 23, 1979 to Watson et al., and U.S. Pat. No. 4,230,688, Oct. 28, 1980 to Rowsell et al. The carboxamides in U.S. Pat. No. 4,136,163 are N-substituted-p-menthane-3-carboxamides, such as N-ethyl-p-menthane-3-carboxamide, commercially available as WS-3 from Wilkinson Sword. The carboxamides of U.S. Pat. No. 4,230,688 are certain acyclic tertiary and secondary carboxamides, such as trimethyl isopropyl butanamide, commercially available as WS-23 from Wilkinson Sword. More preferred for use herein are monomenthyl glutarate, N-ethyl-p-menthane-3-carboxamide, trimethyl isopropyl butanamide and mixtures thereof, more preferably still monomenthyl glutarate, commercially available as Ultracool 2 from IFF (Netherlands). The balance of the cooling composition may be made up of a suitable appropriate carrier, such as water, propylene glycol or a bulk sweetener, described in more detail below. The cooling composition can further comprise a warming agent as described herein provided that the predominant effect is one of cooling.

The medicaments of the present invention may further comprise a warming composition. An essential component of the warming composition is a physiological warming agent. Suitable levels of the warming agent are from about 0.001 to about 10%, preferably from about 0.005 to about 5%, more preferably from about 0.01 to about 1%, more preferably still from about 0.01% to about 0.5% by weight of the throat drop.

Physiological warming agents can be tested for using a modification of the test for cooling agents described above, the test being modified to use benzyl alcohol rather than menthol as the reference sample. Preferred physiological warming agents are those selected from the group consisting of vanillyl alcohol n-butyl ether, vanillyl alcohol n-propyl ether, vanillyl alcohol isopropyl ether, vanillyl alcohol isobutyl ether, vanillyl alcohol n-amino ether, vanillyl alcohol isoamyl ether, vanillyl alcohol n-hexyl ether, vanillyl alcohol methyl ether, vanillyl alcohol ethyl ether, gingerol, shogaol, paradol, zingerone, capsaicin, dihydrocapsaicin, nordihydrocapsaicin, homocapsaicin, homodihydrocapsaicin, ethanol, iso-propyl alcohol, iso-amylalcohol, benzyl alcohol, chloroform, eugenol, cinnamon oil, cinnamic aldehyde, phosphate derivatives thereof, and mixtures thereof. The phosphate derivatives mentioned are those described in WO 97/02273. A commercial example of a suitable warming agent for use herein is Optaheat (Symrise, Germany). The balance of the warming composition may be made up of a suitable appropriate carrier, such as water, propylene glycol or a bulk sweetener, described in more detail below. The warming composition can further comprise a cooling agent as described herein provided that the predominant effect is one of warming.

The medicaments herein can also include a flavouring agent. As used herein, the term ‘flavouring agent’ means those flavour essences and equivalent synthetic ingredients which are added to the flavour composition for the principal purpose of providing flavour to the confectionery product. It excludes warming and cooling agents as described above. Flavouring agents well known in the confectionery art can be added to the flavour compositions of the invention. These flavouring agents can be chosen from synthetic flavouring liquid and/or oils derived from plants leaves, flowers, fruits and so forth, and combinations thereof. Representative flavouring liquids include: artificial, natural or synthetic fruit flavours such as eucalyptus, lemon, orange, banana, grape, lime, apricot and grapefruit oils and fruit essences including apple, strawberry, cherry, orange, pineapple and so forth; bean and nut derived flavours such as coffee, cocoa, cola, peanut, almond and so forth; and root derive flavours such as licorice or ginger. The amount of flavouring agent employed is normally a matter of preference subject to such factors as flavour type, base type and strength desired. In general, amounts up to about 4% by weight are usable with amounts of from about 0.1% to about 1% being preferred.

The medicaments of the present invention may further comprise other active ingredients such as local anaesthetics, antitussives, decongestants and the like. The medicaments may also comprise pH adjusting agents and buffers in order to control the pH and hence the stability of the medicament. Other optional ingredients include organic acids including citric, ascorbic, malic, tartaric acids, or mixtures thereof. Where the medicament is the form of a water-based syrup, the medicament may further comprise thickening agents that impart an increased viscosity to the liquid formulation.

The present invention further provides a composition for modulating oral and/or nasal secretion comprising:
a) a salivating agent according to formula (I): 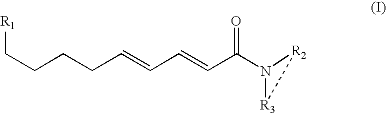
wherein R₁ represents C1-C2 n-alkyl; R₂ is 2-methyl-1-propyl and R₃ is hydrogen, or R₂ and R₃ taken together is a moiety having the formula —(CH₂)ₙ— wherein n is 4 or 5, or mixtures thereof; and
b) a cooling agent comprising menthol, peppermint oil, N-substituted-p-menthane -3-carboxamides, acyclic tertiary and secondary carboxamides, 3-1-menthoxy propan-1,2-diol, monomenthyl glutarate and mixtures thereof.

Without wishing to be bound by theory it is believed that the compositions of the present invention provide improved soothing of oral and/or nasal tissues by a synergistic action of modulating oral and/or nasal secretions and improved cooling and soothing sensation associated with the cooling agent. The two ingredients act together to provide an improved composition that soothes and relieves irritated oral and/or nasal tissues.

Preferably the composition comprises a salivating agent comprising trans-pellitorin. Preferably the compositions comprises from about 0.01% to about 5% salivating agent by weight of the composition, preferably from about 0.01% to about 2%, more preferably from about 0.01% to about 1.5%, even more preferably from about 0.02% to about 1.0%.

Suitable levels of the cooling agent are from about 0.001 to about 10%, preferably from about 0.01 to about 5%, more preferably from about 0.01 to about 2%, more preferably still from about 0.01 to about 0.5% by weight of the composition. The cooling agent preferably comprises monomenthyl glutarate.

The following examples are given to illustrate the compositions and uses according to the invention. However, the invention is not limited thereto.

Sugar-Free Hard Boiled Candy

### EXAMPLE 1

1. Preparation of Premixes

a) MEDICATION PREMIX

| DESCRIPTION | MAN % w/w |
|---|---|
| FLAVOUR | 38.33 |
| LEVOMENTHOL EP | 12.77 |
| MONOMENTHOL GLUTARATE | 24.33 |
| OPTAFLOW (TRANS- | 19.46 |
| PELLITORIN) | |
| ALCOHOL 96% | 5.11 |
| Total | 100.00 |

Combine the above ingredients, heating if required up to a maximum of 20° C. and stir until fully homogeneous.

b) ACESULFAME K/COLOUR SOLUTION

| DESCRIPTION | MAN % w/w |
|---|---|
| WATER | 81.39 |
| ACESULFAME K | 10.47 |
| BETA-CAROTINE 1% CWS | 8.14 |
| E160A | |
| Total | 100.00 |

Add the Acesulfame K and the colour ingredient to the water and mix until homogeneous.

c) XYLITOL SOLUTION

| DESCRIPTION | MAN % w/w |
|---|---|
| WATER | 60.00 |
| XYLITOL | 40.00 |
| Total | 100.00 |

Xylitol is dissolved in water and mixed until homogeneous.

2. Making Process

Isomalt Liquid (93.7%) and xylitol solution (3.8%) are weighed and added to the cooker. The mixture is cooked to 148° C. applying a vacuum of −0.92 bar minimum after cooking. To the hot candy base, the following ingredients are added by weight of the composition: Acesulfame K/colour solution (0.4%), citric acid anhydrous (1.2%), ascorbic acid (0.6%), medication premix (0.4%), aspartame (0.05%) and flavours (2%).

The medicated/coloured candy mass is then transferred onto the cooling belt to cool down the mass. A thin film of trennwax is used to ensure separation of the mass from the cooling belt. The mass is transferred through batch former, rope former and punching equipment to form drops. Talc is used to prevent the mass/drops from sticking to the forming equipment. The drops are cooled down on the cooling belt passing the cooling tunnel to obtain their final consistency for packing.

Sugar-Based Hard Boiled Candy

### EXAMPLE 2

1. Preparation of Premixes

a) MEDICATION PREMIX

| DESCRIPTION | MAN % w/w |
|---|---|
| FLAVOUR | 23.13 |
| LEVOMENTHOL EP | 16.51 |
| MONOMENTHYL GLUTARATE | 30.72 |
| OPTAFLOW (TRANS- | 24.57 |
| PELLITORIN) | |
| ALCOHOL 96% | 5.07 |
| Total | 100.00 |

Combine the ingredients of the medication pre-mix, heating if required up to a maximum of 20° C. and stir until fully homogeneous.

b) COLOUR SOLUTION

| | Man % w/w |
|---|---|
| WATER | 68.182 |
| BETACAROTINE 1% CWS (160A) | 31.818 |
| Total | 100.00 |

To the water add the colour ingredient(s) under stirring at ambient temperature and stir until completely dissolved.

2. Making Process

Water (16.1%), sucrose (48.4%), glucose liquid (36.3%) and an antifoam agent are weighed and added to the cooker. The mixture is cooked to 148° C. applying a vacuum of −0.92 bar minimum after cooking. To the hot candy base, the following ingredients are added (per 100 kg of candy mass): B carotene solution (0.1%), citric acid anhydrous (1.2%), ascorbic acid (0.6%), and medication premix (0.3%) and flavour (2%).

The medicated/coloured candy mass is then transferred onto the cooling belt to cool down the mass. A thin film of trennwax is used to ensure separation of the mass from the cooling belt. The mass is transferred through batch former, rope former and punching equipment to form drops. Talc is used to prevent the mass/drops from sticking to the forming equipment. The drops are cooled down on the cooling belt passing the cooling tunnel to obtain their final consistency for packing

Gelatine Based Gummy

a) Cooked gelatine base:

| DESCRIPTION | MAN % w/w |
|---|---|
| WATER | 16.54% |
| GELATINE 250 BLOOM | 5.26% |
| SUGAR | 45.11% |
| GLUCOSE SYRUP | 33.08% |
| C₁₆-C₁₈ VEGETABLE FAT | 0.01% |
| Total | 100.00 |

Premix the water, gelatine and vegetable fat into a premix vessel. Add sugar and glucose syrup until homogeneous. Cook the mix using a jet cooker at 120-130° C. and a vacuum of 3.2-3.8 bar. Dispense the cooked gelatine base into a finishing vessel, keep at 70-80° C.

b) Gummy finishing:

| DESCRIPTION | MAN % w/w |
|---|---|
| COOKED GELATINE BASE | 95.80% |
| OPTAFLOW (TRANS- | 0.08% |
| PELLITORIN) | |
| MONOMETHYL GLUTARATE | 0.30% |
| FLAVOUR | 0.12% |
| FRUIT JUICE CONCENTRATE | 2.00% |
| CITRIC ACID MONOHYDRATE | 1.50% |
| COLOURANT | 0.20% |
| Total | 100.00 |

Add to cooked gelatine base at 70-80° C. the flavouring, fruit juice concentrate, citric acid and colourant. Mix until fully homogeneous. Transfer to filling line and deposit into pre-moulded starch trays.
Leave the gummies to cure in the starch moulds for 24 hours at 25° C. until gelled, for a harder consistency leave gummies to cure for longer.
Once cured de-mould the gummies and separate from the starch. Polish the gummies in a coating drum with a vegetable wax for a smooth finish.

1. Preparation of Premixes

a) MEDICATION PREMIX

| DESCRIPTION | Man % w/w |
|---|---|
| FLAVOUR | 66.04 |
| MONOMENTHYL GLUTARATE | 18.87 |
| OPTAFLOW (TRANS- | 15.09 |
| PELLITORIN) | |
| Total | 100.00 |

Combine the ingredients of the medication pre-mix, heating if required up to a maximum of 20° C. and stir until fully homogeneous.

b) COLOUR SOLUTION

| | Man % w/w |
|---|---|
| WATER | 95.00 |
| FOOD COLOUR LIGHT GREEN | 5.00 |
| Total | 100.00 |

To the water add the colour ingredient(s) under stirring at ambient temperature and stir until completely dissolved.

2. Making Process

Water (16.1%), sucrose (48.4%), glucose liquid (36.3%) and an antifoam agent are weighed and added to the cooker. The mixture is cooked to 148° C. applying a vacuum of −0.92 bar minimum after cooking. To the hot candy base, the following ingredients are added (per 100 kg of candy mass): light green colour solution (0.1%), citric acid (0.8%), malic acid (0.4%), medication premix (0.5%) and flavour (2.0%).

The medicated/coloured candy mass is then transferred onto the cooling belt to cool down the mass. A thin film of trennwax is used to ensure separation of the mass from the cooling belt. The mass is transferred through batch former, rope former and punching equipment to form drops. Talc is used to prevent the mass/drops from sticking to the forming equipment. The drops are cooled down on the cooling belt passing the cooling tunnel to obtain their final consistency for packing

### EXAMPLE 5

Cough lozenge

| DESCRIPTION | Man % w/w |
|---|---|
| SUCROSE | 62.3 |
| GLUCOSE LIQUID 80% | 43.3 |
| OPTAFLOW (TRANS-PELLITORIN) | 0.08 |
| FLAVOUR | 0.1 |
| LEVOMENTHOL | 0.2 |
| SIMETHICONE EMULSION 30% | 0.0075 |
| ETHANOL | 0.1 |
| VEGETABLE OIL & WAX | 0.025 |
| DEXTROMETHORPHAN BASE | 0.2 |
| HONEY OR/AND HONEY FLAVOUR | 2.6 |
| PURIFIED WATER | UP TO 100 |

### EXAMPLE 6

Cough syrup

| DESCRIPTION | Man % w/w |
|---|---|
| SUCROSE | 36.24 |
| GLUCOSE LIQUID 80% | 36.24 |
| POLYOLS AND GLYCOLS | 12.5 |
| ALCOHOL | 9.8 |
| PRESERVATIVES | 4.1% |
| OPTAFLOW (TRANS-PELLITORIN) | 0.08 |
| ACTIVES | 1.5 |
| FLAVOURS | 0.62 |
| COLOUR | 0.09 |
| PURIFIED WATER | UP TO 100 |

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.
While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for soothing irritated oral or nasal tissue comprising administration of a medicament comprising a salivating agent according to formula (I): 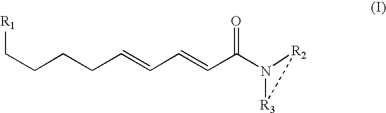
wherein R₁ represents C1-C2 n-alkyl; R₂ is 2-methyl-1-propyl and R₃ is hydrogen, or R₂ and R₃ taken together is a moiety having the formula —(CH₂)ₙ— wherein n is 4 or 5, or mixtures thereof.

2. The method according to claim 1, wherein the salivating agent comprises trans-pellitorin.

3. The method according to claim 1, wherein the medicament comprises from about 0.01% to about 5% of a salivating agent, by weight of the medicament.

4. The method according to claim 3, wherein from about 0.01% to about 2% of the salivating agent, by weight of the medicament, is administered.

5. The method according to claim 4, wherein from about 0.01% to about 1.5% of the salivating agent, by weight of the medicament, is administered.

6. The method according to any claim 5, wherein from about 0.02% to about 1.0% of the salivating agent, by weight of the medicament, is administered.

7. The method according to claim 1, wherein the medicament is in the form of a confectionery.

8. The method according to claim 7, wherein the medicament is in the form of a hard boiled sweet.

9. The method according to claim 1, wherein the medicament further comprises monomenthyl glutarate.

10. A composition for modulating oral or nasal secretion comprising:
a) a salivating agent according to formula (I): 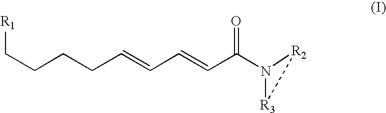
wherein R₁ represents C1-C2 n-alkyl; R₂ is 2-methyl-1-propyl and R₃ is hydrogen, or R₂ and R₃ taken together is a moiety having the formula —(CH₂)ₙ— wherein n is 4 or 5, or mixtures thereof; and
c) a cooling agent selected from the group consisting of menthol, peppermint oil, N-substituted-p-menthane-3-carboxamides, acyclic tertiary and secondary carboxamides, 3-1-menthoxy propan-1,2-diol, monomenthyl glutarate and mixtures thereof.

11. The composition according to claim 10, wherein the composition comprises from about 0.001% to about 10% of the cooling agent, by weight of the composition.

12. The composition according to claim 10, wherein the cooling agent comprises monomenthyl glutarate.

13. The composition according to claim 10, wherein the composition comprises from about 0.01% to about 5% of the salivating agent, by weight of the composition,

14. The composition according to claim 13, wherein the composition comprises from about 0.01% to about 2% of the salivating agent, by weight of the composition.

15. The composition according to claim 14, wherein the composition comprises from about 0.01% to about 1.5% of the salivating agent, by weight of the composition.

16. The composition according to claim 15, wherein the composition comprises from about 0.02% to about 1.0% of the salivating agent, by weight of the composition.

17. The composition according to 10, wherein the salivating agent comprises trans-pellitorin.
